(19) United States
(12) Patent Application Publication
(10) Pub. No.: US 2024/0310373 A1
(43) Pub. Date: Sep. 19, 2024

(54) BIOSENSOR, METHOD OF MAKING THE SAME, AND MOLECULE DETECTION METHOD

(71) Applicant: SOUTHERN TAIWAN UNIVERSITY OF SCIENCE AND TECHNOLOGY, Tainan City (TW)

(72) Inventors: YING-NIEN CHOU, Tainan City (TW); WEI-HAO GONG, Tainan City (TW)

(21) Appl. No.: 18/327,902

(22) Filed: Jun. 2, 2023

(30) Foreign Application Priority Data
Mar. 16, 2023 (TW) 112109686

(51) Int. Cl. G01N 33/543

(52) U.S. Cl. CPC G01N 33/54386

(57) ABSTRACT

A biosensor, a method of making the same, and a molecule detection method for detecting target molecules comprises a heat-shrinkable film. The heat-shrinkable film is made of a thermoplastic material shrinkable when heated and a plurality of antigen molecules are adsorbed to the heat-shrinkable film. The method of making a biosensor involves the steps of: (a) providing a heat-shrinkable film made of a thermoplastic material shrinkable when heated; (b) providing an antigen solution, the antigen solution having a plurality of antigen molecules; and (c) soaking the heat-shrinkable film in the antigen solution for at least 10 minutes. The molecule detection method involves the steps of: (a) providing the biosensor; (b) providing a substance under detection and allowing the substance under detection to come into contact with the biosensor; and (c) detecting a signal outputted by the biosensor upon contact with the substance under detection.

### CROSS-REFERENCE TO RELATED APPLICATION

This non-provisional application claims priority under 35 U.S.C. § 119(a) on Patent Application No. 112109686 filed in Taiwan, R.O.C. on Mar. 16, 2023, the entire contents of which are hereby incorporated by reference.

### BACKGROUND OF THE INVENTION

### 1. Field of the Invention

The present disclosure relates to a biosensor, a method of making the same, and a molecule detection method, and in particular to a biosensor using a heat-shrinkable film as a carrier.

### 2. Description of the Related Art

Biosensors are detection devices developed according to principles of specificity recognition and biological reactions. Nowadays biosensors have broad medical, environmental, and food-related applications.

A biosensor includes a biological recognition element and a signal conversion element. The biological recognition element is designed to come into contact with a substance under detection. The biological recognition element in contact with the substance under detection undergoes a specific binding reaction and thereby generates light signals, thermal signals, electrochemical signals, or the like. Then, the signal conversion element converts the aforesaid signals into outputable signals to detect target molecules in the substance under detection and detect the quantity of the target molecules. The detection sensitivity of conventional biosensors is enhanced in the following ways: increasing the surface area of biosensors to increase antibody adsorption area (see “Modification of a nitrocellulose membrane with cellulose nanofibers for enhanced sensitivity of lateral flow assays: application to the determination of Staphylococcus aureus”, Microchim Acta (2019) 186:831); increasing microstructures on biosensors to increase the quantity of antibodies adsorbed to the biosensors (see “Controlled Hierarchical Architecture in Surface-initiated Zwitterionic Polymer Brushes with Structurally Regulated Functionalities”, Adv. Mater. 2012, 24, 1834-1837).

### BRIEF SUMMARY OF THE INVENTION

Conventional biosensors usually work with a signal-amplifying reagent to render output signals more conspicuous. However, the use of the signal-amplifying reagent incurs detection cost and complicates the detection process. Therefore, it is imperative to provide a biosensor that exhibits high sensitivity and works well without any signal-amplifying reagent.

To achieve the above and other objectives, the present disclosure provides a biosensor, comprising: a heat-shrinkable film made of a thermoplastic material shrinkable when heated; and a plurality of antigen molecules adsorbed to the heat-shrinkable film.

Regarding the biosensor, the thermoplastic material is one selected from the group consisting of polyolefin (POF), polyvinyl chloride, polyethylene terephthalate, and polyethylene terephthalate glycol (PETG).

In an embodiment of the present invention, the thermoplastic material is polyolefin (POF).

In an embodiment of the present invention, the heat-shrinkable film has a rough surface.

In an embodiment of the present invention, the rough surface of the heat-shrinkable film has a roughness of 120 to 500 nm.

In an embodiment of the present invention, the antigen molecules are fibrinogen.

To achieve the above and other objectives, the present disclosure provides a method of making a biosensor, comprising the steps of: (a) providing a heat-shrinkable film made of a thermoplastic material shrinkable when heated; (b) providing an antigen solution, the antigen solution containing a plurality of antigen molecules; and (c) soaking the heat-shrinkable film in the antigen solution for at least 10 minutes.

In an embodiment of the method of making a biosensor of the present invention, the heat-shrinkable film has a rough surface.

The method of making a biosensor further comprises the step of: (d) taking the heat-shrinkable film out of the antigen solution and then soaking the heat-shrinkable film in a blocking solution for at least 10 minutes.

To achieve the above and other objectives, the present disclosure provides a molecule detection method, comprising the steps of: (a) providing the biosensor; (b) providing a substance under detection and allowing the substance under detection to come into contact with the biosensor; and (c) detecting a signal outputted by the biosensor upon contact with the substance under detection.

Therefore, the present disclosure provides a biosensor, a method of making the same, and a molecule detection method and particularly provides a biosensor that exhibits high sensitivity and works well without any signal-amplifying reagent as well as a molecule detection method that renders the biosensor more effective.

### BRIEF DESCRIPTION OF THE DRAWINGS

FIG. 1 is a schematic view of the appearance of a heat-shrinkable film according to an embodiment of the present disclosure.

FIG. 2 illustrates the thermal shrinkage percentage of the heat-shrinkable film according to an embodiment of the present disclosure.

FIG. 3 is a schematic view of the appearance of a heat-shrinkable film according to an embodiment of the present disclosure.

FIG. 4 shows images of the surface of a biosensor under an optical microscope according to an embodiment of the present disclosure.

FIG. 5 shows images of the surfaces of the biosensors under an atomic force microscope according to an embodiment of the present disclosure.

FIG. 6 illustrates the relationship between the detected adsorbance of biosensors made by different processes and the wavelength range of 470-620 nm before shrinkage and after shrinkage.

FIG. 7 shows the Attenuated Total Reflectance-Infrared Fourier Transform Spectrum (ATR-FTIR) of the biosensors according to an embodiment of the present disclosure.

FIG. 8 shows a picture taken of the antibody-bound biosensors of a control group and experimental groups 1-8 before and after their shrinkage in a biosensor detection performance test according to an embodiment of the present disclosure.

FIG. 9 illustrates the detected adsorbance of the antibody-bound biosensors of a control group and experimental groups 1-8 before and after their shrinkage in a biosensor detection performance test within the wavelength range of 470-630 nm according to an embodiment of the present disclosure.

FIG. 10 illustrates the detected adsorbance of the antibody-bound biosensors of a control group and experimental groups 1-8 before and after their shrinkage in a biosensor detection performance test at the wavelength of 540 nm according to an embodiment of the present disclosure.

FIG. 11 is a schematic view of the appearance of a heat-shrinkable film according to an embodiment of the present disclosure.

FIG. 12 shows a picture taken of the antibody-bound biosensors of a control group and experimental groups 1-8 before and after their shrinkage in a biosensor detection performance test according to an embodiment of the present disclosure.

FIG. 13 illustrates the detected adsorbance of the antibody-bound biosensors of a control group and experimental groups 1-8 before and after their shrinkage in a biosensor detection performance test within the wavelength range of 470-620 nm according to an embodiment of the present disclosure.

FIG. 14 illustrates the detected adsorbance of the antibody-bound biosensors of a control group and experimental groups 1-8 before and after their shrinkage in a biosensor detection performance test at the wavelength of 540 nm according to an embodiment of the present disclosure.

FIG. 15 is a schematic view of the appearance of a heat-shrinkable film according to an embodiment of the present disclosure.

FIG. 16 shows a picture taken of the antibody-bound biosensors of a control group and experimental groups 1-8 before and after their shrinkage in a biosensor detection performance test according to an embodiment of the present disclosure.

FIG. 17 illustrates the detected adsorbance of the antibody-bound biosensors of a control group and experimental groups 1-8 before and after their shrinkage in a biosensor detection performance test within the wavelength range of 470-620 nm according to an embodiment of the present disclosure.

FIG. 18 illustrates the detected adsorbance of the antibody-bound biosensors of a control group and experimental groups 1-8 before and after their shrinkage in a biosensor detection performance test at the wavelength of 540 nm according to an embodiment of the present disclosure.

FIG. 19 shows images of the heat-shrinkable film 11 observed under a fluorescence microscope in a fluorescence detection performance test according to an embodiment of the present disclosure.

FIG. 20 illustrates the detected fluorescence signal strength of the biosensors each comprising the heat-shrinkable film 11 in a control group and experimental groups 1-5 in a fluorescence detection performance test within the wavelength range of 530-630 nm according to an embodiment of the present disclosure.

FIG. 21 illustrates the detected fluorescence signal strength of the biosensors each comprising the heat-shrinkable film 11 in a control group and experimental groups 1-5 in a fluorescence detection performance test at the wavelength of 540 nm according to an embodiment of the present disclosure.

FIG. 22 illustrates the detected adsorbance of the biosensor of experimental group 1 before and after shrinkage in a detection performance test within the wavelength range of 470-620 nm in a complicated environment and blood environment according to an embodiment of the present disclosure.

FIG. 23 illustrates the detected adsorbance of the biosensor of experimental group 2 before and after shrinkage in a detection performance test within the wavelength range of 470-620 nm in a complicated environment and blood environment according to an embodiment of the present disclosure.

FIG. 24 illustrates the detected adsorbance of the biosensors of experimental groups 1 and 2 before and after their shrinkage in a detection performance test at the wavelength of 540 nm in a complicated environment and blood environment according to an embodiment of the present disclosure.

### DETAILED DESCRIPTION OF THE INVENTION

Objectives, features, and advantages of the present disclosure are herein illustrated with specific embodiments, depicted with drawings, and described below.

### Method of Making a Biosensor in an Embodiment of the Present Invention

First, the method of making a biosensor comprises providing a heat-shrinkable film made of polyolefin (POF) and then cutting the heat-shrinkable film into one that is round in shape and 1 cm in diameter.

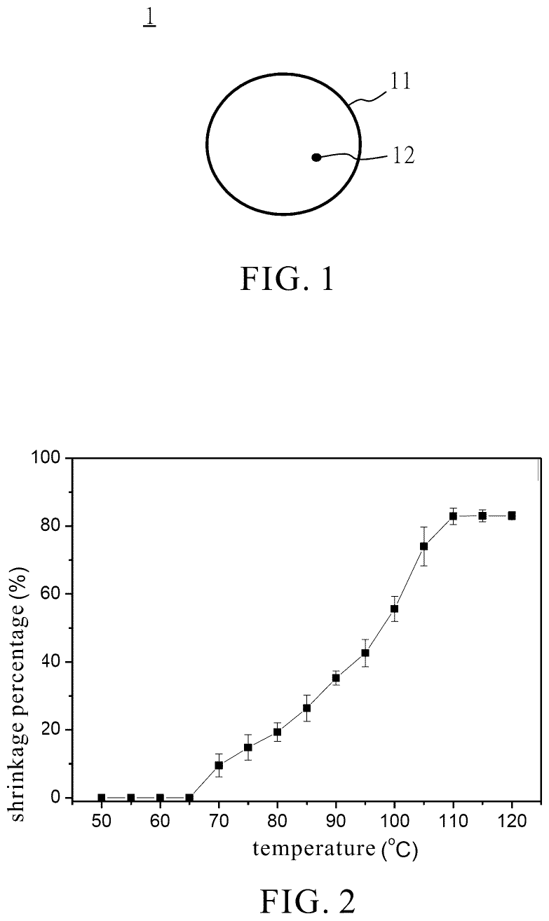

Then, the heat-shrinkable film after cutting is soaked in 1 mL of antigen solution comprising a plurality of fibrinogen molecules. The fibrinogen molecules function as antigens. In the antigen solution, the fibrinogen has a concentration of 10⁶ ng/mL and soaks at an environment temperature of 37° C. (adjustable to, for example, 15-40° C. to meet process requirements) for 30 minutes so that the fibrinogen molecules are adsorbed to the heat-shrinkable film. After the heat-shrinkable film has been soaked in the antigen solution, the heat-shrinkable film is taken out and rinsed with phosphate buffered saline (PBS). Upon completion of the rinsing process, the heat-shrinkable film is soaked in 1 mg/mL bovine serum albumin (BSA) solution to undergo a blocking reaction continuously for 30 minutes. The BSA solution serves as a blocking solution, and the concentration of BSA is adjustable to meet process requirements. Upon completion of the process of its soaking in the antigen solution, the heat-shrinkable film is taken out and rinsed with phosphate buffered saline (PBS). At this point in time, a biosensor 1 of an embodiment has been made. Referring to FIG. 1, the biosensor 1 comprises a heat-shrinkable film 11 and a plurality of fibrinogen molecules 12. The fibrinogen molecules 12 are adsorbed to the heat-shrinkable film 11 (FIG. 1 shows only one of the fibrinogen molecules 12 for exemplary sake).

In an embodiment of the present invention, the heat-shrinkable film 11 is soaked in the blocking solution to undergo the blocking reaction for the sole purpose of further blocking non-specific antigen-antibody binding when the biosensor 1 is in use, but this step can be dispensed with to meet usage criteria or process requirements. In an embodiment, although the blocking solution is a BSA solution, it can be any existing, known blocking solution, such as skim milk.

In an embodiment of the present invention, the heat-shrinkable film 11 is rinsed with PBS to remove any reaction solution otherwise remaining on the heat-shrinkable film 11, but this step can be dispensed with to meet usage criteria or process requirements.

In an embodiment, although the heat-shrinkable film 11 is made of polyolefin (POF), the heat-shrinkable film 11 can also be made of any other existing, known thermoplastic materials shrinkable when heated, such as, for example, polyvinyl chloride (PVC), polyethylene terephthalate (PET), and polyethylene terephthalate glycol (PETG). In an embodiment, although the heat-shrinkable film 11 is cut into one that has a specific shape and a specific size to meet experimental requirements, the heat-shrinkable film 11 can be designed to come in any other shapes or sizes.

In an embodiment, the heat-shrinkable film 11 is soaked in the antigen solution and the blocking solution for 30 minutes, but the duration of soaking the heat-shrinkable film 11 in the antigen solution and the blocking solution is adjustable to meet process requirements and other criteria. The fibrinogen molecules 12 can be adsorbed to the heat-shrinkable film 11 if the heat-shrinkable film 11 is soaked in the antigen solution for 10 minutes or longer. The heat-shrinkable film 11 can undergo the blocking reaction if the heat-shrinkable film 11 is soaked in the blocking solution for 10 minutes or longer. The heat-shrinkable film 11 can be soaked in the antigen solution and the blocking solution for, for example, 10, 12, 14, 16, 18, 20, 22, 24, 26 or 28 minutes or for a time period longer than 30 minutes.

In an embodiment, although the fibrinogen functions as an antigen, any other types of antigen molecules can be adsorbed to the heat-shrinkable film 11 to make the biosensor 1.

### Thermal Shrinkage Percentage Test Conducted on the Heat-Shrinkable Film 11 of an Embodiment of the Present Invention

First, the heat-shrinkable film 11 of an embodiment is provided. Then, the thermal shrinkage percentage of the heat-shrinkable film 11 is determined with a test designed in accordance with ASTM D2732 standard regulations to determine the free linear thermal shrinkage percentage of plastic thin-films and thin plates. The test is described below. First, a temperature-controlled oil bath pot is provided and filled with room temperature water, and a thermocouple electronic thermometer for measuring water temperature in the temperature-controlled oil bath pot is provided. Then, the water in the temperature-controlled oil bath pot is heated to 50° C. Next, the heat-shrinkable film 11 is put in the water in the temperature-controlled oil bath pot at 50° C. to heat up the heat-shrinkable film 11. The heat-shrinkable film 11 is heated up continuously at 50° C. until the heat-shrinkable film 11 stops shrinking. Then, the aforesaid thermal shrinkage percentage test is carried out again to heat up the water in the temperature-controlled oil bath pot to, for example, 55° C., 60° C., 65° C., 70° C., 75° C., 80° C., 85° C., 90° C., 95° C., 100° C., 105° C., 110° C., 115° C. and 120° C. After that, at the temperatures, the aforesaid thermal shrinkage percentage test is carried out to measure the shrinkage rates of the heat-shrinkable film 11 at the respective temperatures.

The results of the test are shown in FIG. 2, indicating that the shrinkage rate of the heat-shrinkable film 11 has a substantial linear correlation with the heating temperature and thus is increasing when the heat-shrinkable film 11 is heated up within the temperature range of 65 to 110° C., but the heat-shrinkable film 11 has a shrinkage rate that levels off (i.e., ending up round in shape with a diameter of 0.25 cm) when the heat-shrinkable film 11 is heated up within the temperature range of 110 to 120° C. The heating temperature at which the test is conducted to evaluate the characteristics of the biosensor 1 is set to 120° C. to apply the test results and ensure the stability of subsequent test results. However, in other situations, the heating temperature can be set to any temperature other than 120° C. to suit the material characteristics of the heat-shrinkable film 11 or meet usage needs, so as to attain different thermal shrinkage percentages.

Using the heat-shrinkable film 11 of an embodiment of the present invention in fluorescence detection performance test.

The test aims to assess the relationship between shrinkage ratio and fluorescence detection signal enhancement when the heat-shrinkable film 11 is used in fluorescence detection.

First, six heat-shrinkable films 11 (round in shape and 1 cm in diameter) are provided.

Then, five out of the six heat-shrinkable films 11 are soaked continuously in 0.1 wt % rhodamine solution dissolved in methanol for 10 seconds. Rhodamine is a dye that can emit fluorescence, allowing the heat-shrinkable films 11 to undergo fluorescent staining through the soaking process. Upon completion of the process of soaking the heat-shrinkable films 11 in the rhodamine solution, the stained heat-shrinkable films 11 are taken out of the rhodamine solution. The non-stained heat-shrinkable film 11 serves as a control group. The five stained heat-shrinkable films 11 serve as experimental groups 1-5, respectively.

The control group and experimental group 1 are directly observed under a Nexcope NIB410, an inverted fluorescence microscope with phase contrast, and have their images photographically captured, resulting in the finding that experimental group 1 exhibits a shrinkage ratio of 0% (1 cm² in area). Next, experimental groups 2-5 are sequentially placed in an oven at 120° C. in order to be heated up for a predetermined time period, allowing the stained heat-shrinkable films 11 to undergo thermal shrinkage. The heating duration is 5 seconds for experimental group 2, 20 seconds for experimental group 3, 60 seconds for experimental group 4, and 120 seconds for experimental group 5. Upon completion of the heating process of experimental groups 2-5, experimental groups 2-5 are taken out of the oven; meanwhile, the shrinkage ratio is found to be 25% (0.8 cm²) in experimental group 2, 50% (0.65 cm²) in experimental group 3, 75% (0.45 cm²) in experimental group 4, and 100% (0.25 cm²) in experimental group 5. Then, experimental groups 2-5, which have undergone thermal shrinkage, are observed under a fluorescence microscope and have their images photographically captured.

Finally, the fluorescence signal strength in the observed images of the control group and experimental groups 1-5 is detected with a PerkinElmer LS-55 fluorescence spectroscopy, resulting in the finding of a fluorescence signal value of 0 in the control group at a wavelength of 585 nm, a fluorescence signal value of 72.99944 in experimental group 1 at the wavelength of 585 nm, a fluorescence signal value of 81.38357 in experimental group 2 at the wavelength of 585 nm, a fluorescence signal value of 93.11732 in experimental group 3 at the wavelength of 585 nm, a fluorescence signal value of 167.66825 in experimental group 4 at the wavelength of 585 nm, and a fluorescence signal value of 566.31045 in experimental group 5 at the wavelength of 585 nm. Then, fluorescence signal multiplying factors for experimental groups 2-5 are calculated by setting the standard fluorescence signal value to the fluorescence signal value in experimental group 1 at the wavelength of 585 nm (The fluorescence signal of Rhodamine at 585 nm indicates the greatest adsorption wavelength).

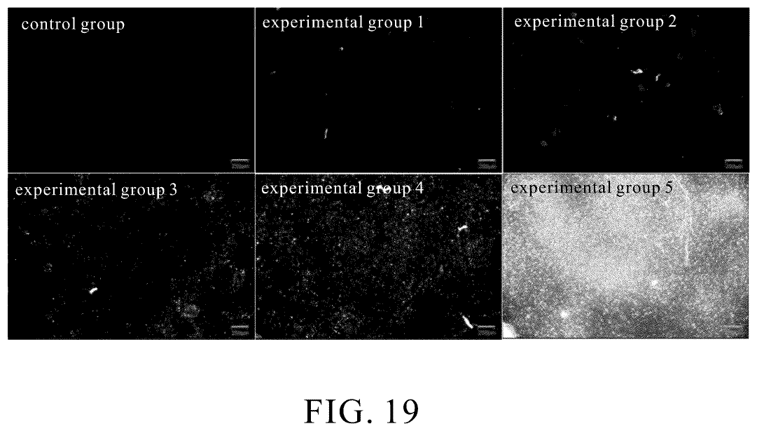

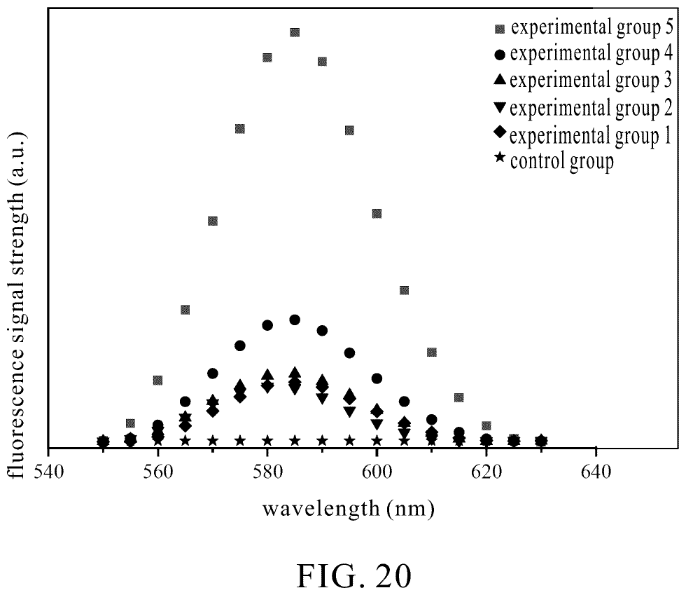

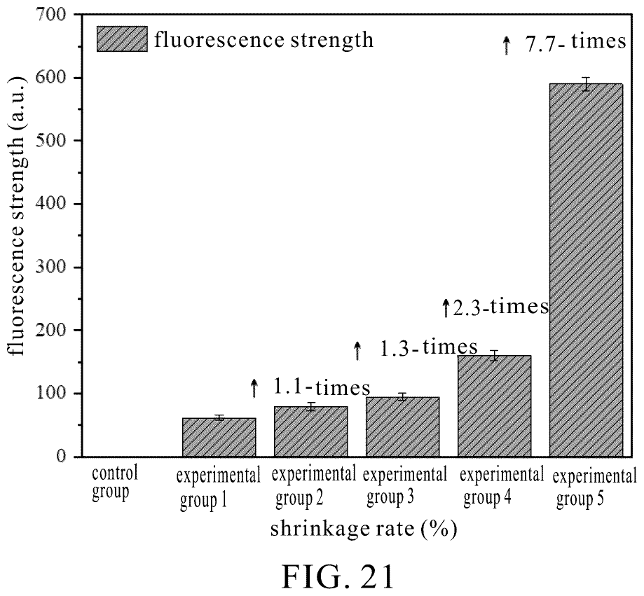

The results of the test are shown in FIGS. 19-21. As shown in FIG. 19, the observed image of experimental group 1 (not having shrunk at all) displays a small amount of fluorescence points only, whereas the fluorescence quantity in the observed images of experimental groups 2-5 increases with shrinkage rate. As shown in FIG. 20, experimental group 5 has the greatest fluorescence signal value. Referring to FIG. 21, with the standard fluorescence signal value being set to the fluorescence signal value in experimental group 1, the fluorescence signal value multiplying factors for experimental groups 2-5 are 1.1, 1.3, 2.3 and 7.8, respectively.

### Method of Making a Biosensor According to an Embodiment of the Present Invention

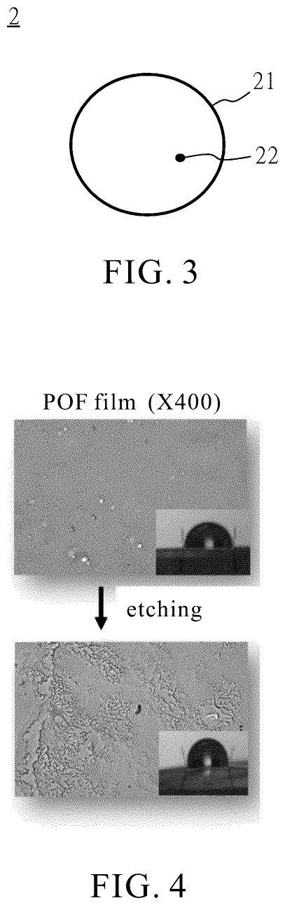

The method of making a biosensor 2 and its structure in an embodiment are substantially the same as those in disclosed in previous embodiments. As shown in FIG. 3, the biosensor 2 comprises a heat-shrinkable film 21 and a plurality of antigen molecules 22 adsorbed to the heat-shrinkable film 21 (FIG. 3 shows only one of the fibrinogen molecules 22 for exemplary sake). The major distinguishing technical feature of this embodiment is: The heat-shrinkable film 21 of the biosensor 2 undergoes an etching step before being soaked in an antigen solution. The etching step which the heat-shrinkable film 21 undergoes is described below.

After the heat-shrinkable film 21 has been cut into one that is round in shape and 1 cm in diameter, the heat-shrinkable film 21 is soaked continuously in a toluene solution with a concentration of 99.5% for 30 minutes so that the surface of the heat-shrinkable film 21 undergoes an etching reaction to turn into a rough surface. Upon completion of the process of soaking the heat-shrinkable film 21 in the toluene solution, the heat-shrinkable film 21 is taken out, rinsed with an acetone solution, and rinsed with PBS, so as to be soaked in a fibrinogen antigen solution in order to make the biosensor 2.

The etching reaction takes, for example, 10, 12, 14, 16, 18, 20, 22, 24, 26 or 28 minutes or lasts for a time period longer than 30 minutes. The toluene solution for use with the etching reaction can be replaced with any other known etching solution whereby the heat-shrinkable film 21 develops a rough surface.

### Surface Roughness Comparison Test Conducted on Biosensor 2 of an Embodiment of the Present Invention

The difference in surface roughness between the heat-shrinkable film 21 before etching and the heat-shrinkable film 21 after etching is evaluated with a surface roughness comparison test as described below. First, a heat-shrinkable film 21 that has not yet undergone an etching reaction is provided to serve as a control group, and another heat-shrinkable film 21 that has undergone an etching reaction is provided to serve as an experimental group. The heat-shrinkable film 21 of the control group and the heat-shrinkable film 21 of the experimental group are identical in size and shape.

Then, the heat-shrinkable film 21 of the control group and the heat-shrinkable film 21 of the experimental group are observed under a Nexcope NIB410, an inverted fluorescence microscope with phase contrast, at 400× magnification to observe the surfaces of the heat-shrinkable films 21 of the control group and experimental group. FIG. 4 shows images of the surfaces of the heat-shrinkable films 21 of the control group and experimental group under the optical microscope, resulting in the finding of a flat, even surface of the heat-shrinkable film 21 of the control group and an uneven surface of the heat-shrinkable film 21 of the experimental group.

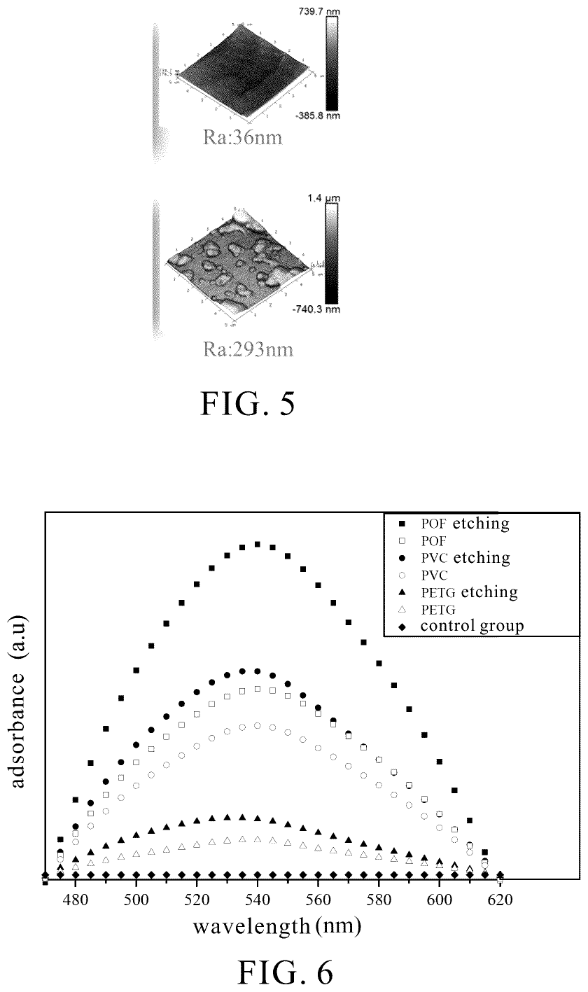

Finally, the heat-shrinkable films 21 of the control group and experimental group are placed under an atomic force microscope (Bruker CA-1115) to further observe the zones observed under the optical microscope and have their images photographically captured, and then the surface roughness displayed in the images of the heat-shrinkable films 21 of the control group and experimental group observed under the atomic force microscope undergoes quantitative analysis. FIG. 5 shows images of the surfaces of the heat-shrinkable films 21 of the control group and experimental group under the atomic force microscope. The surface roughness of the heat-shrinkable films 21 of the control group and the experimental group is calculated, with built-in software of the atomic force microscope and according to the atomic force microscopic images in FIG. 5, to be 36 nm and 293 nm, respectively, indicating that etching is the cause of the much greater surface roughness of the heat-shrinkable film 21 of the experimental group. In an embodiment, the surface roughness of the biosensor 2 measures 293 nm but may be adjusted to fall within the range of 250 to 350 nm to meet process or usage needs or changed to any other surface roughness value to meet process or usage needs. The surface roughness of the biosensor 2 can be adjusted by adjusting the duration of soaking the heat-shrinkable film 21 in an etching solution (i.e., the duration of the etching reaction).

### Test for Comparing Biosensors Made by Different Processes in Terms of Antigen Adsorption Performance

First, given the method of making a biosensor in previous embodiments, the antigen concentration is kept at 1 mg/ml to effectuate surface adsorption and thereby make the biosensor (made of POF and etched) of experimental group 1. Then, given the method of making a biosensor in previous embodiments, the antigen concentration is kept at 1 mg/ml to effectuate surface adsorption and thereby make the biosensor (made of POF but not etched) of experimental group 2. Next, given the method of making a biosensor in an embodiment, the antigen concentration is kept at 1 mg/ml to effectuate surface adsorption and thereby make the biosensor (made of PVC and etched) of experimental group 3. Then, given the method of making a biosensor in an embodiment without an etching process, the antigen concentration is kept at 1 mg/ml to effectuate surface adsorption and thereby make the biosensor (made of PVC but not etched) of experimental group 4. After that, given the method of making a biosensor in an embodiment, the antigen concentration is kept at 1 mg/ml to effectuate surface adsorption and thereby make the biosensor (made of PETG and etched) of experimental group 5. Then, given the method of making a biosensor in an embodiment without an etching process, the antigen concentration is kept at 1 mg/ml to effectuate surface adsorption and thereby make the biosensor (made of PETG but not etched) of experimental group 6. To perform the test, the biosensor of the control group directly employs the heat-shrinkable film 21 that has been cut out to become round in shape and 1 cm in diameter but has not been soaked in an antigen solution.

Then, an antibody solution for allowing the biosensors of the experimental groups 1-6 and control group to undergo a reaction in the test is prepared according to the method of preparing an antibody solution for use with the detection performance test conducted on the biosensors in an embodiment. The antibody solution comprises gold nanoparticles that anti-fibrinogen antibodies are bound to. Then, the process of the detection performance test conducted on the biosensors in an embodiment is carried out to allow the biosensors of experimental groups 1-6 and control group in the test to react with the antibody solution, and the biosensors of experimental groups 1-6 and the control group in the test are heated up at 120° C. for 10 minutes to enable the complete shrinkage of the biosensors of the experimental groups and the control group. Finally, the adsorbance of the biosensors (having shrunk) of the control group and the experimental groups 1-6 within the wavelength range of 470-620 nm is detected with a UV-Vis spectrophotometer.

FIG. 6 illustrates an antigen adsorption performance comparison test for the biosensors of the experimental groups 1-6 and the control group. The findings of the test are described below. Experimental groups 1-6 exhibit greater signal strength than the control group. The non-etched biosensor in an embodiment exhibits enhanced signal strength, and its signals can be easily detected by an instrument. The signal strength of the biosensor (made of POF and etched) of experimental group 1 at the wavelength of 540 nm is 1.8 times greater than the signal strength of the biosensor (made of POF but not etched) of experimental group 2 at the wavelength of 540 nm. The signal strength of the biosensor (made of PVC and etched) of experimental group 3 is 1.4 times greater than the signal strength of the biosensor (made of PVC but not etched) of experimental group 4. The signal strength of the biosensor (made of PETG and etched) of experimental group 5 is 1.6 times greater than the signal strength of the biosensor (made of PETG but not etched) of experimental group 6. Therefore, the test shows that the biosensors demonstrate enhanced sensitivity when etched.

Analysis of surface molecules of biosensor 2 in an embodiment of the present invention.

A heat-shrinkable film which no antigen is adsorbed to is provided to serve as a control group (having been cut into one that is round in shape and 1 cm in diameter). The biosensor 2 which has not been heated up is provided to serve as experimental group 1 (having been cut into one that is round in shape and 1 cm in diameter). The biosensor 2 which has been heated up is provided to serve as experimental group 2 (having undergone thermal shrinkage and being round in shape and 0.25 cm in diameter).

Then, the biosensors of the control group, experimental groups 1 and 2 are placed in the Attenuated Total Reflectance-Infrared Fourier Transform Spectroscopy (ATR-FTIR) (Model No.: Perkin Elmer Frontier) to analyze the surface adsorption spectrums of the control group and the experimental groups 1 and 2 and thereby analyze the surface molecule types of the control group and the experimental groups 1 and 2.

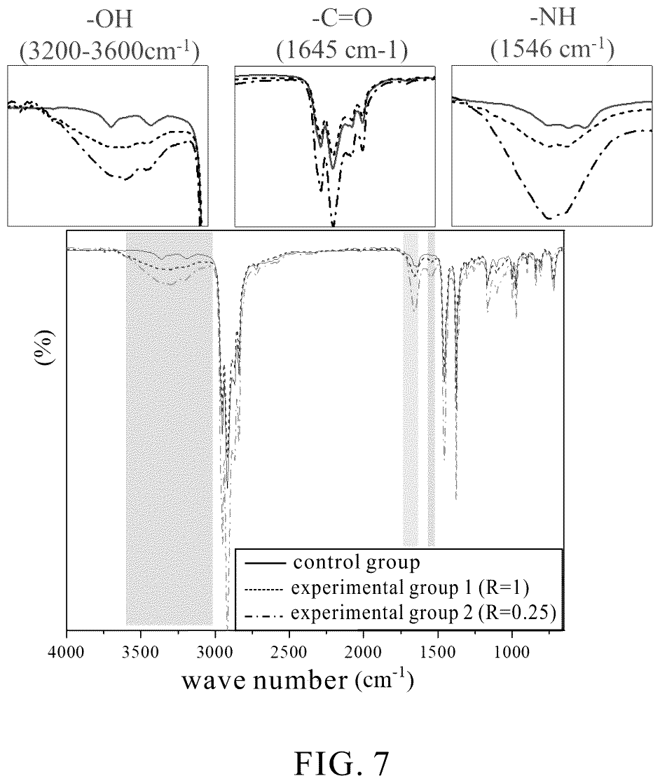

FIG. 7 shows that peaks occur at wave numbers 1546 cm⁻¹, 1645 cm⁻¹, and 3200-3600 cm⁻¹ of the spectral waveform of experimental group 1 in the spectrum of ATR-FTIR of this analysis, where the peak at wave number 1546 cm⁻¹ corresponds to the presence of amino group (—NH), the peak at wave number 1645 cm⁻¹ corresponds to the presence of carbonyl group (—C═O), and the peak at wave number 3200-3600 cm⁻¹ corresponds to the presence of hydroxyl group (—OH). The peaks are feature adsorption peaks of protein antigen molecules, i.e., signals that are indicative of antigen molecules and are obtained in experimental group 1. The analysis result shows that the antigen molecules 22 in the biosensor 2 of experimental group 1 are adsorbed to the heat-shrinkable film 21.

Peaks occur at wave numbers 1546 cm⁻¹, 1645 cm⁻¹, and 3200-3600 cm⁻¹ of the spectral waveform of experimental group 2, and the peaks of experimental group 2 are higher than the peaks of experimental group 1. That indicates the following: the antigen molecules 22 in the biosensor 2 that has undergone thermal shrinkage are adsorbed to the heat-shrinkable film 21; and the biosensor 2 that has undergone thermal shrinkage provides stronger signals than the biosensor 2 that has not undergone thermal shrinkage.

No conspicuous waveform is present at the peak (corresponding to the presence of amino group (—NH)) at wave number 1546 cm⁻¹ and the peak (corresponding to the presence of hydroxyl group (—OH)) at wave number 3200-3600 cm⁻¹ in the control group, indicating that the control group lacks the adsorption of protein antigen molecules and thus fails to detect the presence of a feature adsorption peak of protein.

### Detection Performance Test for Biosensor in an Embodiment of the Present Invention

First, the biosensors of the control group and the experimental groups 1-8 are provided. The biosensor of the control group comprises the heat-shrinkable film 21 having been cut into one that is round in shape and 1 cm in diameter in order to undergo the test and not having been soaked in an antigen solution. The biosensors of the experimental groups 1-8 are made by the method of making the biosensor 2 in an embodiment and by adjustably setting the concentration of the antigen solution to 10⁶ ng/mL for experimental group 1, 10⁵ ng/mL for experimental group 2, 10⁴ ng/mL for experimental group 3, 10³ ng/mL for experimental group 4, 10² ng/mL for experimental group 5, 10¹ ng/mL for experimental group 6, 1 ng/mL for experimental group 7, and 0 ng/mL for experimental group 8.

Then, an antibody solution for evaluating the detection performance of the biosensors is provided. The antibody solution simulates a substance under detection to be detected by the biosensors. The method of preparing the antibody solution is described below.

First, a gold nanoparticle solution (with a concentration of 150 ppm) comprising gold nanoparticles with a particle diameter of 40 nm is provided. The pH of the gold nanoparticle solution is adjusted to 7.74. Then, 1 mL of the gold nanoparticle solution is added to a 10 mL centrifugal tube. Next, 10 μL of anti-fibrinogen antibody (purchased from Arigo Biolaboratories) solution (with a concentration of 1.5 mg/ml) is added to the gold nanoparticle solution to prepare an anti-fibrinogen antibody—gold nanoparticle solution. After that, the centrifugal tube containing the anti-fibrinogen antibody—gold nanoparticle solution is placed in a rotary shaking incubator such that a rotation and mixing process takes place at room temperature at 70 rpm for 30 minutes, allowing the anti-fibrinogen antibodies to be bonded to the gold nanoparticles.

Afterward, 100 μL of 5% BSA solution (with a concentration of 50 mg/mL) is added to the anti-fibrinogen antibody—gold nanoparticle solution to prepare a BSA-antibody—gold nanoparticle solution. Then, the centrifugal tube containing the BSA-antibody—gold nanoparticle solution is placed in a rotary shaking incubator such that a rotation and mixing process takes place at room temperature at 70 rpm for 30 minutes, allowing a blocking reaction to occur between the BSA and the gold nanoparticles.

Then, upon completion of the blocking reaction, the BSA-antibody—gold nanoparticle solution is placed in a centrifuge and rotated at 4° C. and at a rotation speed of 9600 rcf for 20 minutes to undergo centrifugal separation, allowing the BSA-antibody—gold nanoparticle solution to undergo centrifugal separation and layering.

Upon completion of the centrifugal separation process, the gold nanoparticles in the BSA-antibody—gold nanoparticle solution precipitate and settle at the bottom of the centrifugal tube. Then, the supernatant is taken out of the centrifugal tube. Next, 1 mL of 1% BSA solution is added to the centrifugal tube such that the otherwise precipitated gold nanoparticles get dissolved in the BSA solution again. After that, the aforesaid step of performing centrifugal separation and taking out the supernatant is carried out again. Finally, 500 μL of PBS and 1% BSA solution (with a concentration of 10 mg/mL) is added to the centrifugal tube such that the gold nanoparticles get dissolved in the newly introduced solution to form an antibody solution containing the gold nanoparticles which anti-fibrinogen antibodies are bonded to.

After the biosensors of the control group and the experimental groups 1-8 and the antibody solution have been provided, the biosensors of the control group and the experimental groups 1-8 are placed in a 24-well plate. Then, 40 μL of the antibody solution is added to each of the wells of the 24-well plate holding the biosensors of the control group and the experimental groups 1-8 and left to stand 30 minutes at 37° C. such that antigen molecules on the biosensors of the control group and the experimental groups 1-8 undergo a specific antigen-antibody binding reaction with the anti-fibrinogen antibody/gold nanoparticles in the antibody solution. At this point in time, with the fibrinogen being bonded to the anti-fibrinogen antibodies, the gold nanoparticles which the anti-fibrinogen antibodies are bonded to are adsorbed to the biosensors; thus, the gold nanoparticles cause the biosensors to manifest a color change. The color that the gold nanoparticles take on enables the biosensors to output optical signals when detected by an instrument. After the biosensors of the control group and the experimental groups 1-8 have stood still for 30 minutes, the antibody solution is completely removed from the wells holding the biosensors of the control group and the experimental groups 1-8. Next, the biosensors of the control group and the experimental groups 1-8 are rinsed with PBS and then taken out of the wells, so as for the biosensors to be left to stand until they turn dry.

After the biosensors of the control group and the experimental groups 1-8 have turned dry, the biosensors of the control group and the experimental groups 1-8 are placed in a UV-Vis spectrophotometer such that optical signals outputted from the biosensors of the control group and the experimental groups 1-8 can be detected to determine the adsorbance of the biosensors. The test aims to detect and determine the adsorbance of the biosensors of the control group and the experimental groups 1-8 within the wavelength range of 470-630 nm. Upon completion of the detection and determination process, the biosensors of the control group and the experimental groups 1-8 are taken out and heated up at 120° C. for 10 minutes, so as for the biosensors of the control group and the experimental groups 1-8 to undergo shrinkage thoroughly. Finally, the biosensors of the control group and the experimental groups 1-8 which have undergone shrinkage thoroughly are placed in the UV-Vis spectrophotometer to detect and determine the adsorbance of the biosensors of the control group and the experimental groups 1-8 again.

### Conducting Detection Performance Test on Biosensor by the Process

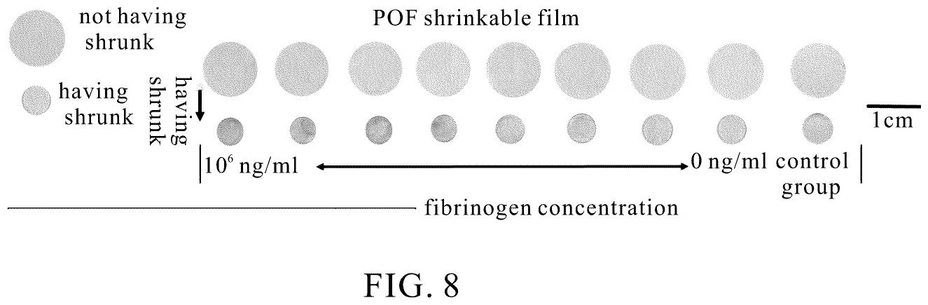

FIG. 8 shows a picture taken of the antibody-bound biosensors of a control group and the experimental groups 1-8 before and after their shrinkage in the biosensor detection performance test according to an embodiment of the present disclosure, showing findings as follows: the biosensors of the control group and the experimental groups 1-8 do not exhibit any noticeable color change before their thermal shrinkage; and the biosensors of the experimental groups 1-4 exhibit noticeable color changes after thermal shrinkage.

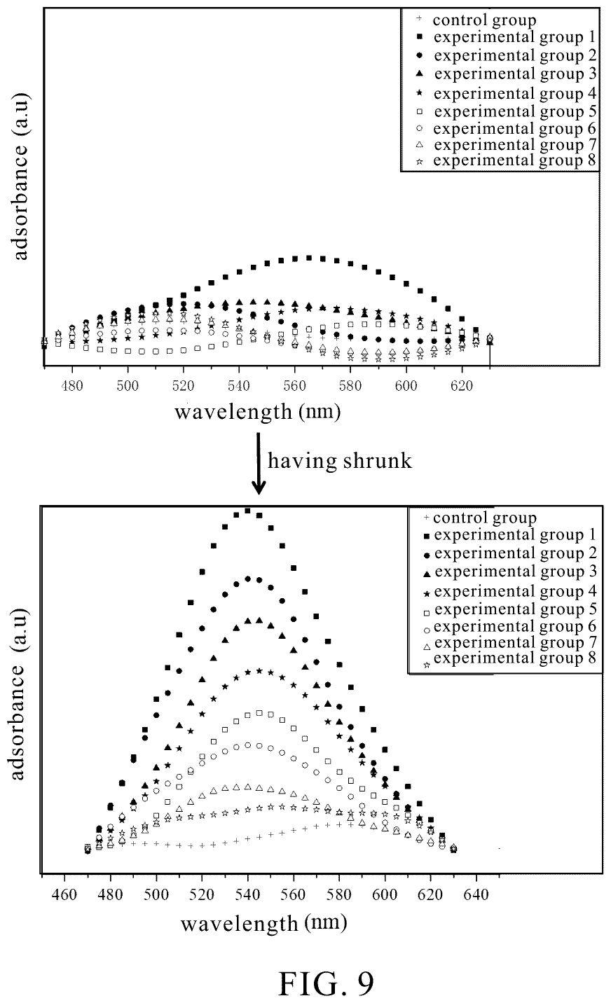

Referring to FIG. 9, the detection and determination of the adsorbance of the biosensors of the control group and the experimental groups 1-8 within the wavelength range of 470-630 nm results in the findings as follows: the biosensors of the experimental groups 1-7 exhibit noticeable higher adsorbance after shrinkage than before shrinkage within the majority of the wavelength range; and the biosensors of the experimental groups 1-7 exhibit the greatest increase in the signal strength of the adsorbance at the wavelength of 540 nm. Afterward, the difference in the signal strength between the biosensors of the control group and the experimental groups 1-8 before their shrinkage and the biosensors of the control group and the experimental groups 1-8 after their shrinkage is analyzed according to the adsorbance at the wavelength of 540 nm. Data related to the adsorbance of the biosensors of the control group and the experimental groups 1-8 before and after their shrinkage at the wavelength of 540 nm is shown in Table 1 below.

TABLE 1
adsorbance of biosensors of the control group and the experimental groups
1-8 before and after their shrinkage at the wavelength of 540 nm

| | experi- | experi- | experi- | experi- | experi- | experi- | experi- | experi- | |
|---|---|---|---|---|---|---|---|---|---|
| | mental | mental | mental | mental | mental | mental | mental | mental | |
| | group | group | group | group | group | group | group | group | control |
| | 1 | 2 | 3 | 4 | 5 | 6 | 7 | 8 | group |
| before | 0.963998 | 0.5321 | 0.290547 | 0.04983 | 0.030 | 0.02587 | 0.04366 | 0.04436 | 0.00511 |
| shrinkage | | | | | | | | | |
| after | 7.2274 | 5.96556 | 5.17858 | 4.63661 | 3.4466 | 1.89309 | 1.10912 | 0.69797 | 0.00381 |
| shrinkage | | | | | | | | | |

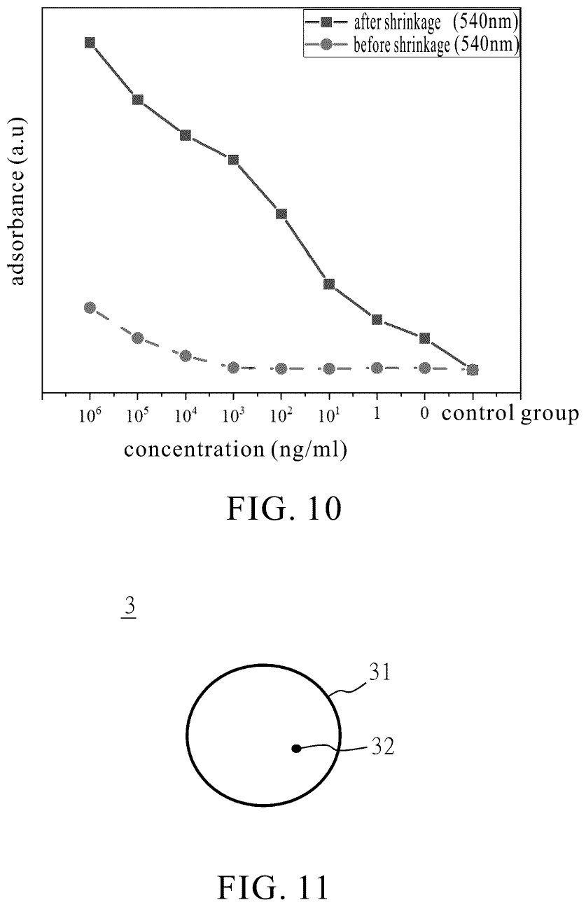

Referring to FIG. 10, the biosensor of the control group does not undergo any antigen-antibody binding reaction and thus does not generate any signal, allowing it to exhibit background adsorbance. As shown in FIG. 10, since the biosensor of the control group does not generate any signal, the adsorbance of the biosensor of the control group appears as the same background adsorbance before and after shrinkage.

The background adsorbance of the biosensor of experimental group 5 (with an antigen concentration of 10² ng/mL) before shrinkage is determined to be equal to that of the biosensor of experimental group 8, and thus no antigen-antibody binding reaction is detected. The background adsorbance of the biosensors of experimental groups 6-7 with an even lower antigen concentration is also determined to be equal to that of the biosensor of experimental group 8. However, after their shrinkage, the biosensors of experimental groups 5-7 exhibit adsorbance higher than the background adsorbance and the adsorbance of the biosensor of experimental group 8 (considering that no antigen molecules are incorporated into the biosensor of experimental group 8, the enhanced signals shown in FIG. 10 are also background noise). Thus, after their thermal shrinkage, the biosensors of experimental groups 5-7 demonstrate further convergence of their optical signals, allowing the UV-Vis spectrophotometer to detect the optical signals of the biosensors of experimental groups 5-7.

Prior to thermal shrinkage, the optical signals of the biosensors of experimental groups 1-4 (with an antigen concentration ranging from 10⁶ to 10³ ng/mL) can be detected by the UV-Vis spectrophotometer. After thermal shrinkage, the signal strength of the biosensors of experimental groups 1-4 increases by signal strength multiplying factors calculated with the equation as follows: (adsorbance of experimental groups 1-7 after shrinkage—adsorbance of experimental group 8 after shrinkage)/(adsorbance of experimental groups 1-7 before shrinkage—adsorbance of experimental group 8 before shrinkage). Since experimental group 8 has an antigen concentration of 0 ng/mL, the adsorbance of experimental group 8 is regarded as a background value to deduct. The signal strength of experimental group 1 after shrinkage is 7.1 times greater than it is before shrinkage. The signal strength of experimental group 2 after shrinkage is 10.8 times greater than it is before shrinkage. The signal strength of experimental group 3 after shrinkage is 18.2 times greater than it is before shrinkage. The signal strength of experimental group 4 after shrinkage is 720 times greater than it is before shrinkage. The data indicates that, after their thermal shrinkage, the biosensors not only manifest enhanced signal strength but also achieve the effect of enhanced sensitivity (i.e., lowering an instrument detection limit). Furthermore, as suggested by the data, the greatest signal enhancement rate can be obtained by adjustably setting the antigen concentration for use with the biosensor 2 of an embodiment in the process to 10³ ng/mL and then allowing the biosensor 2 to undergo thermal shrinkage.

### Detection Performance Test Conducted on Biosensors in a Complicated Environment and Blood Environment According to an Embodiment of the Present Invention

First, the biosensors of experimental groups 1 and 2 are provided. The biosensors of experimental groups 1 and 2 are made with the method of making the biosensor 2 according to a previous embodiment and by adjusting the composition of the antigen solution. A mixture of fibrinogen and bovine serum albumin (BSA) is soaked in the antigen solution for experimental group 1, with both the concentration of fibrinogen and the concentration of bovine serum albumin (BSA) being 1 mg/ml, at a soaking environment temperature of 37° C., and for a soaking duration of 30 minutes, allowing the biosensor of experimental group 1 to simulate the detection performance of the biosensor 2 in a complicated environment. The antigen solution in which the biosensor of experimental group 2 is soaked is human serum. Blood is drawn from the body of a healthy adult. Then, the blood sample is rotated at 3000 rpm for 10 minutes to undergo centrifugal separation. Upon completion of the centrifugal separation, the supernatant serum is collected to function as an antigen solution. The biosensor of experimental group 2 is soaked in the antigen solution at a soaking environment temperature of 37° C. for a soaking duration of 30 minutes to simulate the detection performance of the biosensor 2 in a blood environment.

Then, a specific antigen-antibody binding reaction step in the detection performance test for the biosensors is carried out to enable the biosensors of experimental groups 1 and 2 to undergo a specific antigen-antibody binding reaction with anti-fibrinogen antibody/gold nanoparticles in the antibody solution. Upon completion of the reaction, the biosensors of experimental groups 1 and 2 are rinsed with PBS and then left to stand until they turn dry.

Afterward, the biosensors of experimental groups 1 and 2, which have undergone the specific antigen-antibody binding reaction, are placed in the UV-Vis spectrophotometer to detect optical signals outputted from the biosensors of experimental groups 1 and 2 and thereby determine their adsorbance according to the adsorbance detection step in the detection performance test for the biosensors. In the test, the adsorbance of the biosensors of experimental groups 1 and 2 within the wavelength range of 470-620 nm is determined. Then, the biosensors of experimental groups 1 and 2 are taken out and heated up at 120° C. for 10 minutes to attain thorough shrinkage of the biosensors of experimental groups 1 and 2. Finally, the post-shrinkage biosensors of experimental groups 1 and 2 are placed in the UV-Vis spectrophotometer to determine the adsorbance of the biosensors of experimental groups 1 and 2 once again. After the adsorbance of the biosensors of experimental groups 1 and 2 before and after their shrinkage has been determined, the signal multiplying factors for the biosensors of experimental groups 1 and 2 at the wavelength of 540 nm after shrinkage relative to they are before shrinkage are calculated.

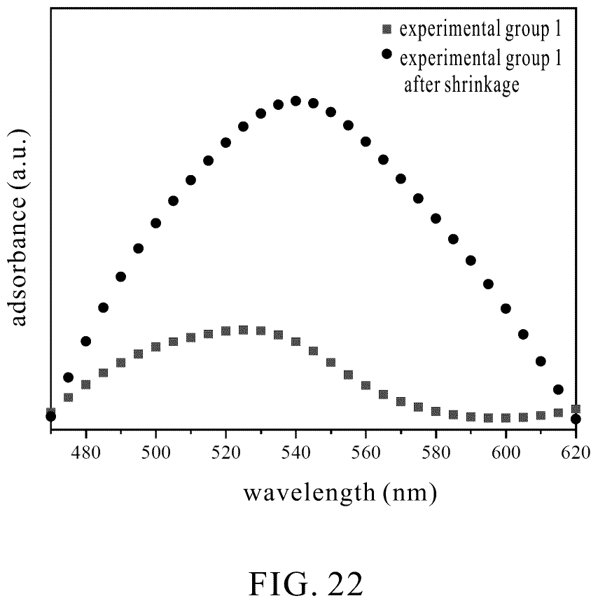

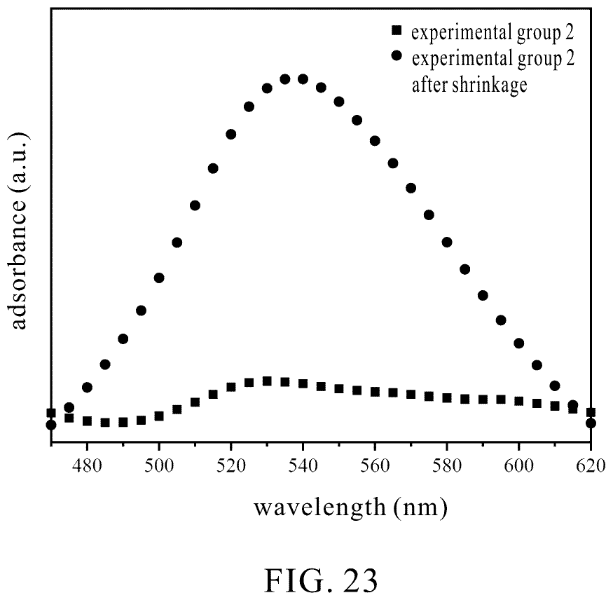

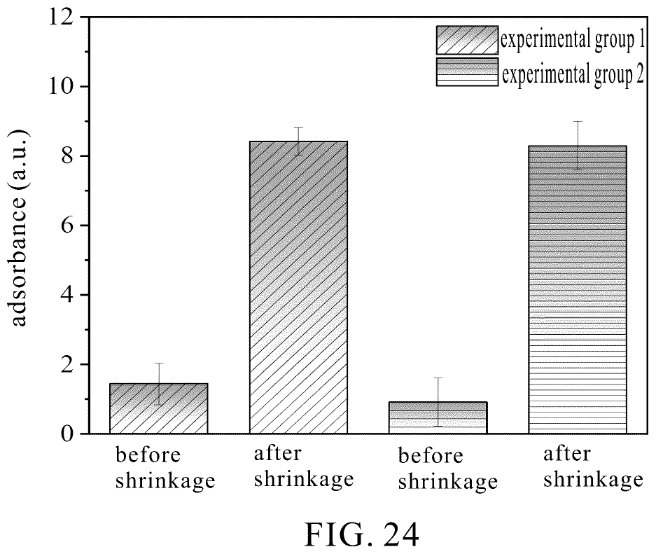

The results of the test are shown in FIGS. 22-24. As shown in FIG. 22 and FIG. 23, the detection and determination of the adsorbance of the biosensors of experimental groups 1 and 2 within the wavelength range of 470-620 nm results in the findings as follows: signals can be detected from the biosensors of experimental groups 1 and 2 before shrinkage; the biosensors of experimental groups 1 and 2 exhibit noticeable higher adsorbance after shrinkage than before shrinkage within the majority of the wavelength range; and the biosensors of experimental groups 1 and 2 exhibit the greatest increase in the signal strength of the adsorbance at the wavelength of 540 nm. The findings show that the biosensor of an embodiment is applicable to detection in a complicated environment and blood environment. Referring to FIG. 24, experimental group 1 exhibits an adsorbance of 1.44095 at the wavelength of 540 nm before shrinkage and an adsorbance of 8.41638 at the wavelength of 540 nm after shrinkage, whereas experimental group 2 exhibits an adsorbance of 0.9123 at the wavelength of 540 nm before shrinkage and an adsorbance of 8.28778 at the wavelength of 540 nm after shrinkage. Therefore, the adsorbance of experimental group 1 at 540 nm after shrinkage is 5.84 times greater than it is before shrinkage, whereas the adsorbance of experimental group 2 at 540 nm after shrinkage is 9.08 times greater than it is before shrinkage.

### Method of Making a Biosensor According to an Embodiment of the Present Invention

The method of making a biosensor 3 and its structure in an embodiment are substantially the same as those in previous embodiments. As shown in FIG. 11, the biosensor 3 comprises a heat-shrinkable film 31 and a plurality of antigen molecules 32 adsorbed to the heat-shrinkable film 31 (FIG. 11 shows only one of the fibrinogen molecules 32 for exemplary sake.) The major distinguishing technical features of an embodiment are described below. In this embodiment, the heat-shrinkable film 31 of the biosensor 3 is made of PETG instead of POF, and etching is carried out with sodium hydroxide (NaOH) with a concentration of 25 wt %. In this embodiment, the surface roughness of the biosensor 3 is changed by an etching process to 450 nm from the pre-etching surface roughness value of 80 nm but may be adjusted to fall within the range of 400 to 500 nm to meet process or usage needs or changed to any other surface roughness value to meet process or usage needs.

### Detection Performance Test for Biosensor in an Embodiment of the Present Invention

First, the biosensors of the control group and the experimental groups 1-8 are provided. The biosensor of the control group comprises the heat-shrinkable film 21 having been cut into one that is round in shape and 0.785 cm² in area in order to undergo the test and not having been soaked in an antigen solution. The biosensors of the experimental groups 1-8 are made by the method of making the biosensor 3 in previous embodiments and by adjustably setting the concentration of the antigen solution to 10⁶ ng/mL for experimental group 1, 10⁵ ng/mL for experimental group 2, 10⁴ ng/mL for experimental group 3, 10³ ng/mL for experimental group 4, 10² ng/mL for experimental group 5, 10¹ ng/mL for experimental group 6, 1 ng/mL for experimental group 7, and 0 ng/mL for experimental group 8.

Then, the detection performance of the biosensors of the control group and the experimental groups 1-8 in the test is evaluated according to the detection performance test process of the biosensor of another embodiment. In the test, the biosensors of the control group and the experimental groups 1-8 each have an area of 0.3925 cm² after shrinkage.

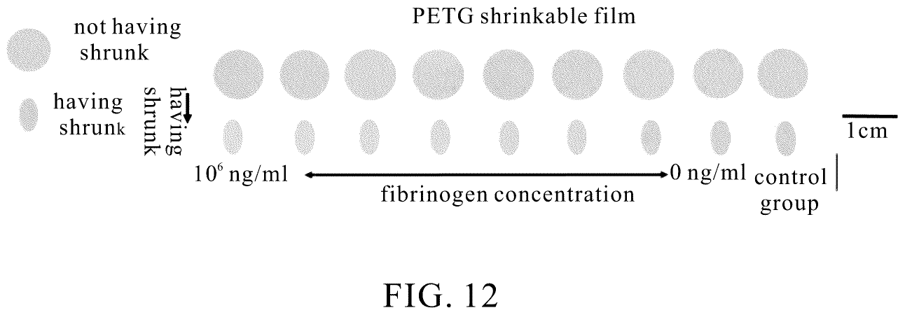

FIG. 12 shows a picture taken of the antibody-bound biosensors of a control group and experimental groups 1-8 before and after their shrinkage in a biosensor detection performance test according to an embodiment of the present disclosure. The biosensors of the control group and the experimental groups 1-8 do not exhibit any noticeable color change before their thermal shrinkage.

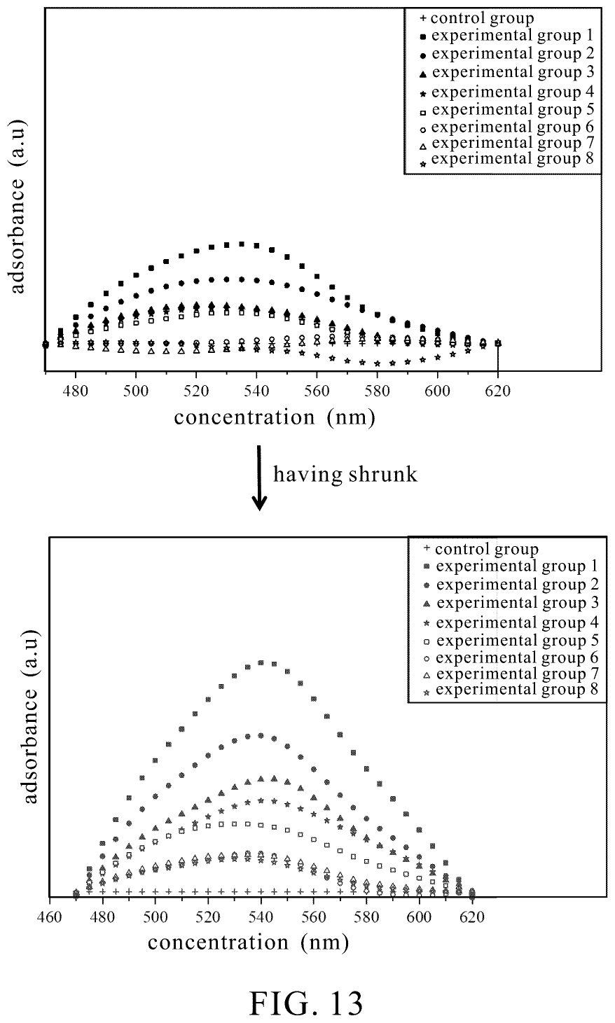

Referring to FIG. 13, the detection and determination of the adsorbance of the biosensors of the control group and the experimental groups 1-8 within the wavelength range of 470-620 nm results in the findings as follows: the biosensors of the experimental groups 1-7 exhibit noticeable higher adsorbance after shrinkage than before shrinkage within the majority of the wavelength range; and the biosensors of the experimental groups 1-7 exhibit the greatest increase in the signal strength of the adsorbance at the wavelength of 540 nm. Afterward, the difference in the signal strength between the biosensors of the control group and the experimental groups 1-8 before their shrinkage and the biosensors of the control group and the experimental groups 1-8 after their shrinkage is analyzed according to the adsorbance at the wavelength of 540 nm. Data related to the adsorbance of the biosensors of the control group and the experimental groups 1-8 before and after their shrinkage at the wavelength of 540 nm is shown in Table 2 below.

TABLE 2
Adsorbance of biosensors of the control group and the experimental groups
1-8 before and after their shrinkage at the wavelength of 540 nm.

| | experi- | experi- | experi- | experi- | experi- | experi- | experi- | experi- | |
|---|---|---|---|---|---|---|---|---|---|
| | mental | mental | mental | mental | mental | mental | mental | mental | |
| | group | group | group | group | group | group | group | group | control |
| | 1 | 2 | 3 | 4 | 5 | 6 | 7 | 8 | group |
| before | 2.062108 | 1.187589 | 0.636025 | 0.296504173 | 0.181988 | 0.13441 | 0.14261 | 0.14282 | 0 |
| shrinkage | | | | | | | | | |
| after | 7.67161 | 6.54478 | 5.7737 | 3.6661 | 2.64227 | 1.77412 | 1.74814 | 1.52989 | 0 |
| shrinkage | | | | | | | | | |

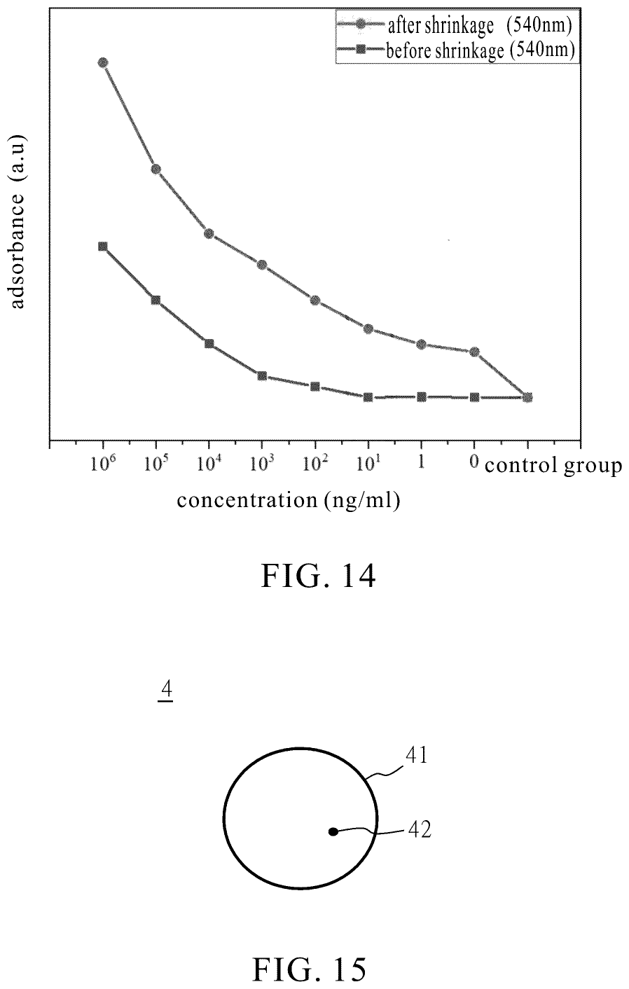

Referring to FIG. 14, the biosensor of the control group does not undergo any antigen-antibody binding reaction and thus does not generate any signal, allowing it to exhibit background adsorbance. As shown in FIG. 14, since the biosensor of the control group does not generate any signal, the adsorbance of the biosensor of the control group appears as the same background adsorbance before and after shrinkage.

The background adsorbance of the biosensor of experimental group 6 (with an antigen concentration of 10¹ ng/mL) before shrinkage is determined to be equal to that of the biosensor of experimental group 8, and thus no antigen-antibody binding reaction is detected. The background adsorbance of the biosensors of experimental group 7 with an even lower antigen concentration is also determined to be equal to that of the biosensor of experimental group 8. However, after their shrinkage, the biosensors of experimental groups 6-7 exhibit adsorbance higher than the background adsorbance and the adsorbance of the biosensor of experimental group 8 (considering that no antigen molecules are incorporated into the biosensor of experimental group 8, the enhanced signals shown in FIG. 14 are also background noise). Thus, after their thermal shrinkage, the biosensors of experimental groups 6-7 demonstrate further convergence of their optical signals, allowing the UV-Vis spectrophotometer to detect the optical signals of the biosensors of experimental groups 6-7.

Prior to thermal shrinkage, the optical signals of the biosensors of experimental groups 1-5 (with an antigen concentration ranging from 10⁶ to 10² ng/mL) can be detected by the UV-Vis spectrophotometer. After thermal shrinkage, the signal strength of the biosensors of experimental groups 1-5 in an embodiment increases by signal strength multiplying factors calculated with the same equation as in previous embodiments. The signal strength of experimental group 1 after shrinkage is 3.2 times greater than it is before shrinkage. The signal strength of experimental group 2 after shrinkage is 4.8 times greater than it is before shrinkage. The signal strength of experimental group 3 after shrinkage is 8.6 times greater than it is before shrinkage. The signal strength of experimental group 4 after shrinkage is 13.9 times greater than it is before shrinkage. The signal strength of experimental group 5 after shrinkage is 28.4 times greater than it is before shrinkage. The data indicates that, after their thermal shrinkage, the biosensors not only manifest enhanced signal strength but also achieve the effect of enhanced sensitivity (i.e., lowering an instrument detection limit).

As suggested by the results of the test conducted on the biosensors according to an embodiment, even if the heat-shrinkable films of the biosensors are made of PETG instead of POF, the effect of enhancing signal strength and enhancing sensitivity (i.e., lowering an instrument detection limit) will still be achieved.

### Method of Making a Biosensor According to an Embodiment of the Present Invention

The method of making a biosensor 4 and its structure in an embodiment are substantially the same as those in previous embodiments. As shown in FIG. 15, the biosensor 4 comprises a heat-shrinkable film 41 and a plurality of antigen molecules 42 adsorbed to the heat-shrinkable film 41 (FIG. 15 shows only one of the fibrinogen molecules 42 for exemplary sake). The major distinguishing technical features of this embodiment are described below. The heat-shrinkable film 41 of the biosensor 4 is made of PVC instead of POF. The etching process is carried out with acetone with a concentration of 99.5 wt %. In this embodiment, the surface roughness value of the biosensor 4 is changed by an etching process to 145 nm from the pre-etching surface roughness value of 10 nm but may be adjusted to fall within the range of 120 to 200 nm to meet process or usage needs or changed to any other surface roughness value to meet process or usage needs.

### Detection Performance Test for Biosensor in an Embodiment of the Present Invention

First, the biosensors of the control group and the experimental groups 1-8 are provided. The biosensor of the control group comprises the heat-shrinkable film 21 having been cut into one that is round in shape and 0.785 cm² in area in order to undergo the test and not having been soaked in an antigen solution. The biosensors of the experimental groups 1-8 are made by the method of making the biosensor 4 in an embodiment and by adjustably setting the concentration of the antigen solution to 10⁶ ng/mL for experimental group 1, 10⁵ ng/mL for experimental group 2, 10⁴ ng/mL for experimental group 3, 10³ ng/mL for experimental group 4, 10² ng/mL for experimental group 5, 10¹ ng/mL for experimental group 6, 1 ng/mL for experimental group 7, and 0 ng/mL for experimental group 8. In the test, the biosensors of the control group and the experimental groups 1-8 each have an area of 0.2748 cm² after shrinkage.

Then, the detection performance of the biosensors of the control group and the experimental groups 1-8 in the test is evaluated according to the detection performance test process of the biosensor of previous embodiments.

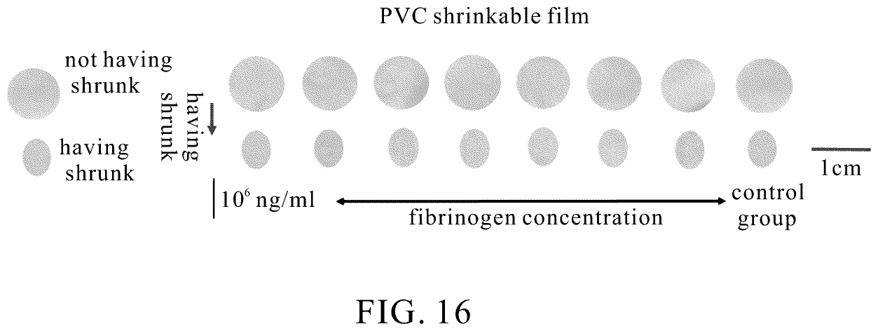

FIG. 16 shows a picture taken of the antibody-bound biosensors of the control group and the experimental groups 1-8 before and after their shrinkage in the biosensor detection performance test according to embodiment 4 of the disclosure. Prior to their thermal shrinkage, the biosensors of the control group and experimental groups 1-8 do not display any noticeable color.

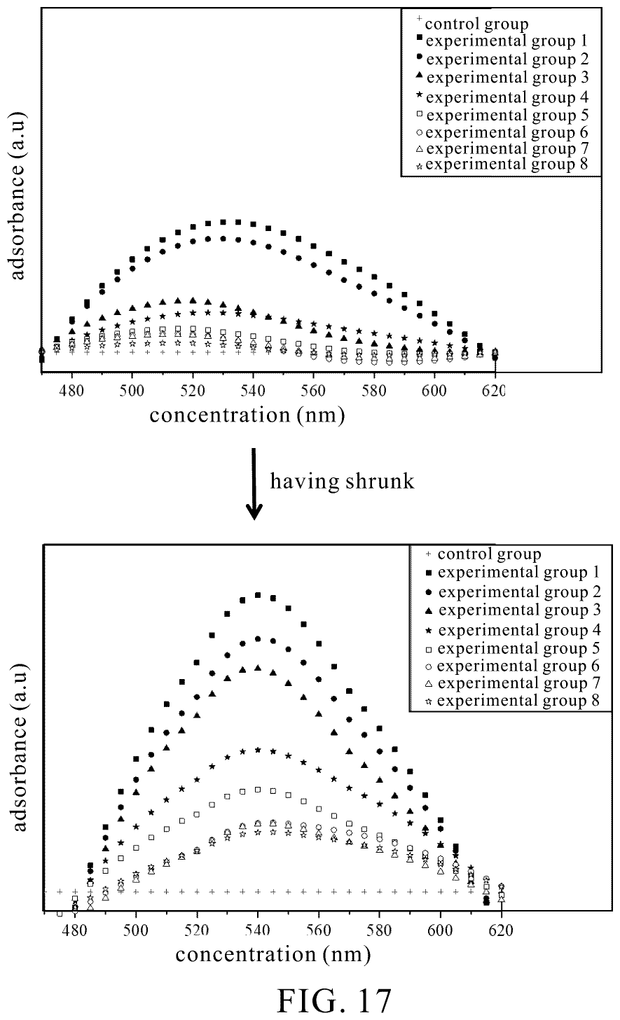

Referring to FIG. 17, the detection and determination of the adsorbance of the biosensors of the control group and the experimental groups 1-8 within the wavelength range of 470-620 nm results in the findings as follows: the biosensors of experimental groups 1-7 exhibit noticeable higher adsorbance after shrinkage than before shrinkage within the majority of the wavelength range; and the biosensors of experimental groups 1-7 exhibit the greatest increase in the signal strength of the adsorbance at the wavelength of 540 nm. Afterward, the difference in the signal strength between the biosensors of the control group and the experimental groups 1-8 before their shrinkage and the biosensors of the control group and the experimental groups 1-8 after their shrinkage is analyzed according to the adsorbance at the wavelength of 540 nm. Data related to the adsorbance of the biosensors of the control group and experimental groups 1-8 before and after their shrinkage at the wavelength of 540 nm is shown in Table 3 below.

TABLE 3
Adsorbance of biosensors of the control group and the experimental groups
1-8 before and after their shrinkage at the wavelength of 540 nm

| | experi- | experi- | experi- | experi- | experi- | experi- | experi- | experi- | |
|---|---|---|---|---|---|---|---|---|---|
| | mental | mental | mental | mental | mental | mental | mental | mental | |
| | group | group | group | group | group | group | group | group | control |
| | 1 | 2 | 3 | 4 | 5 | 6 | 7 | 8 | group |
| before | 2.835967 | 1.369959 | 0.4146 | 0.264290085 | 0.11114 | 0.033009 | 0.029787 | 0.05 | 0 |
| shrinkage | | | | | | | | | |
| after | 7.74049 | 5.27804 | 3.78867 | 3.07017 | 2.24543 | 1.28708 | 1.22821 | 1.05417 | 0 |
| shrinkage | | | | | | | | | |

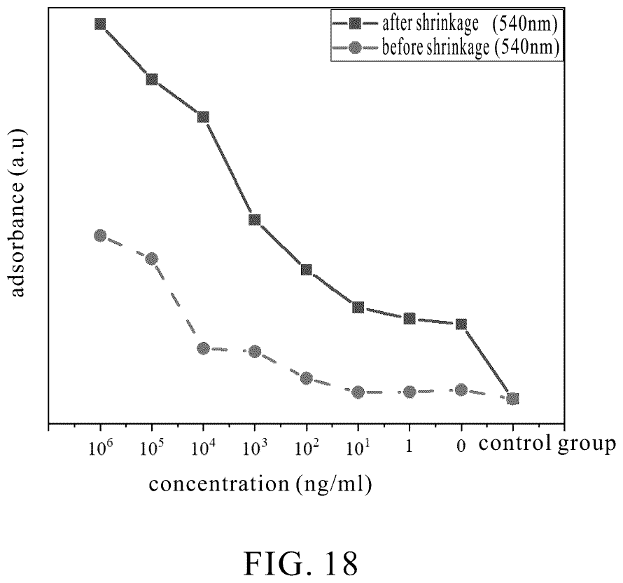

Referring to FIG. 18, the biosensor of the control group does not undergo any antigen-antibody binding reaction and thus does not generate any signal, allowing it to exhibit background adsorbance. As shown in FIG. 18, since the biosensor of the control group does not generate any signal, the adsorbance of the biosensor of the control group appears as the same background adsorbance before and after shrinkage.

The background adsorbance of the biosensor of experimental group 6 (with an antigen concentration of 10¹ ng/mL) before shrinkage is determined to be equal to that of the biosensor of experimental group 8. The background adsorbance of the biosensor of experimental group 7 with an even lower antigen concentration is also determined to be equal to that of the biosensor of experimental group 8.

Prior to thermal shrinkage, the optical signals of the biosensors of experimental groups 1-5 (with an antigen concentration ranging from 10⁶ to 10² ng/mL) can be detected by the UV-Vis spectrophotometer. After thermal shrinkage, the signal strength of the biosensors of experimental groups 1-5 in this embodiment increases by signal strength multiplying factors calculated with the same equation as previous embodiments. The signal strength of experimental group 1 after shrinkage is 2.4 times greater than it is before shrinkage. The signal strength of experimental group 2 after shrinkage is 3.2 times greater than it is before shrinkage. The signal strength of experimental group 3 after shrinkage is 7.5 times greater than it is before shrinkage. The signal strength of experimental group 4 after shrinkage is 9.4 times greater than it is before shrinkage. The signal strength of experimental group 5 after shrinkage is 19.5 times greater than it is before shrinkage. The data indicates that, after their thermal shrinkage, the biosensors not only manifest enhanced signal strength but also achieve the effect of enhanced sensitivity (i.e., lowering an instrument detection limit).

As suggested by the results of the test conducted on the biosensors according to this embodiment, even if the heat-shrinkable films of the biosensors are made of PVC instead of POF, the effect of enhancing signal strength and enhancing sensitivity (i.e., lowering an instrument detection limit) will still be achieved.

As suggested by the experimental results about the biosensors in embodiments, heat-shrinkable films made of different materials can serve as appropriate carriers for the biosensors, and the effect of enhancing signal strength and enhancing sensitivity (i.e., lowering an instrument detection limit) can be achieved by the thermal shrinkage of the heat-shrinkable films. Furthermore, the antigens for use in embodiments are fibrinogens. However, according to the principle of Western Blotting, the antigens adsorbed to heat-shrinkable films can be any other type of existing, known antigens for use in an antibody antigen binding reaction and thus are not restricted to fibrinogens. Moreover, the antigen concentration for use with the biosensors can be adjusted as needed as long as their optical signals are detectable with an instrument.

A molecule detection method for use with the biosensors in embodiments is further provided. First, the biosensors in embodiments are provided. Then, a substance under detection is provided, allowing the substance under detection to come into contact with the biosensors. If the substance under detection contains target molecules, the target molecules will undergo a specific antigen-antibody binding reaction with the antigens in the biosensors. Thus, the biosensors are capable of capturing the target molecules. Next, the biosensors which have come into contact with the substance under detection are placed in a detection instrument to detect signals outputted by the biosensors upon contact with the substance under detection and thereby determine whether the substance under detection contains the target molecules. Upon a determination of the presence of the target molecules, the content of the target molecules in the substance under detection is further analyzed.

As mentioned before, the biosensors' carriers are heat-shrinkable films which antigen molecules are adsorbed to. The heat-shrinkable films undergo a thermal shrinkage reaction and thereby allow all the antigen molecules scattered across the heat-shrinkable films and all the target molecules bonded to the antigen molecules to converge. Therefore, not only is the signal strength enhanced to enable an instrument to detect the signals of the biosensors ((i.e., lowering the instrument's detection limit), but the biosensors also amplify signals through thermal shrinkage, dispensing with any signal-amplifying reagent. Furthermore, the heat-shrinkable films, i.e., the carriers for the biosensors, incur low cost, and thus the biosensors incur low manufacturing cost. Moreover, the biosensors are advantageously compact in operation and easy to carry. In addition, the biosensors each comprise a heat-shrinkable film and antigen molecules and thus manifest structural simplicity, ease of production, ease of detection, ease of use, and feasible applicability to detection in a complicated environment and blood environment. Last but not least, the surface roughness of the heat-shrinkable films of the biosensors is augmented by an etching process, allowing more antigen molecules to be adsorbed to the heat-shrinkable films of the biosensors.

The invention is disclosed above by preferred embodiments. However, persons skilled in the art should understand that the embodiments are illustrative of the invention only, but shall not be interpreted as restrictive of the scope of the invention. Hence, all equivalent modifications and replacements made to the aforesaid embodiments shall be deemed falling within the scope of the claims of the invention. Accordingly, the legal protection for the invention shall be defined by the appended claims.

What is claimed is:

1. A biosensor, comprising:
a heat-shrinkable film made of a thermoplastic material shrinkable when heated; and
a plurality of antigen molecules adsorbed to the heat-shrinkable film.

2. The biosensor of claim 1, wherein the thermoplastic material is one selected from the group consisting of polyolefin (POF), polyvinyl chloride, polyethylene terephthalate, and polyethylene terephthalate glycol (PETG).

3. The biosensor of claim 2, wherein the thermoplastic material is polyolefin (POF).

4. The biosensor of claim 1, wherein the heat-shrinkable film has a rough surface.

5. The biosensor of claim 4, wherein the rough surface of the heat-shrinkable film has a roughness of 120 to 500 nm.

6. The biosensor of claim 1, wherein the antigen molecules are fibrinogen.

7. A method of making a biosensor, comprising the steps of:
(a) providing a heat-shrinkable film made of a thermoplastic material shrinkable when heated;
(b) providing an antigen solution, the antigen solution comprising a plurality of antigen molecules; and
(c) soaking the heat-shrinkable film in the antigen solution for at least 10 minutes.

8. The method of making a biosensor according to claim 7, wherein the heat-shrinkable film has a rough surface.

9. The method of making a biosensor according to claim 7, further comprising the step of:
(d) taking the heat-shrinkable film out of the antigen solution and then soaking the heat-shrinkable film in a blocking solution for at least 10 minutes.

10. A molecule detection method, comprising the steps of:
(a) providing the biosensor of claim 1;
(b) providing a substance under detection and allowing the substance under detection to come into contact with the biosensor; and
(c) detecting a signal outputted by the biosensor upon contact with the substance under detection.
